(19) United States
(12) Patent Application Publication
(10) Pub. No.: US 2004/0193270 A1
(43) Pub. Date: Sep. 30, 2004

(54) Implantable bone graft

(75) Inventors: Thomas M. DiMauro, Southboro, MA (US); Hassan Serhan, South Easton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuyAcroMed, Inc., Raynham, MA

(21) Appl. No.: 10/405,062

(22) Filed: Mar. 31, 2003

(51) Int. Cl. A61F 2/44

(52) U.S. Cl. 623/17.11

(57) ABSTRACT

An intervertebral fusion device, a method of making the intervertebral fusion device, and a method of using the intervertebral fusion device to promote fusion between two consecutive vertebrae in a patient is described. The intervertebral fusion device has an intervertebral fusion cage that has a load bearing wall, and a porous matrix adjacent to the load bearing wall. The load bearing wall of the fusion cage has a greater density than the internal porous matrix. The open pores of the porous matrix define an inner surface to which one or more agents that promote bone growth are attached.

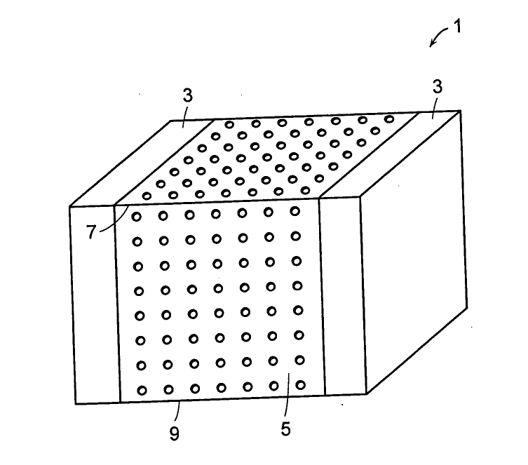

### BACKGROUND OF THE INVENTION

Implantable intervertebral fusion devices are routinely used by surgeons to treat degenerative disc disease, discongenic lower back pain, spondylolisthesis, ruptured discs due to injury and other spinal conditions. Fusion devices are used to keep adjacent vertebrae in the correct spaced apart position while bone growth takes place to complete the fusion of the adjacent vertebrae. Typically, intervertebral fusion devices are hollow cages with side walls made from stainless steel, cobalt or titanium alloy which provide strength to support intervertebral forces. The hollow space between the side walls is usually filled with bone graft material that is either provided by the patient (autogenous) or provided by a third party donor (allogenous).

Unfortunately, the metallic supporting frame of the prior art fusion cages is not osteoconductive and therefore does not form a strong mechanical attachment to a patient's bone tissue. This can lead to graft necrosis, poor fusion and poor stability. In addition, the prior art fusion cages must be filled with autologous bone graft material or allograft bone material. However, the supply of autologous bone material is limited and significant complications can occur from the bone harvesting procedure. Moreover, the patient must undergo two separate incisions which may increase the pain and recuperation time of the patient. Allograft bone material is also in limited supply and carries a risk of disease transmission.

In view of the problems discussed above, a sugnificant need exist for further improvement in the design of spinal fusion devices.

### SUMMARY OF THE INVENTION

The instant invention relates to an intervertebral fusion device, a method of making the intervertebral fusion device and a method of using the intervertebral fusion device to promote fusion between two consecutive vertebrae in a patient. The intervertebral fusion device has an intervertebral fusion cage that has a load bearing wall, and a porous matrix adjacent to the load bearing wall. In a preferred embodiment, the porous matrix is integrally bound to the load bearing wall. The load bearing wall of the fusion cage has a higher compression strength than the internal porous matrix. The open pores of the porous matrix define an inner surface to which one or more agents that promote bone growth are attached. In one embodiment, the agent that promotes bone growth is analogous and is present in a concentration of about 5 times to about 30 times greater than the concentration found in the patient's bone marrow aspirate or platelet rich plasma. Preferably, the agent is mesenchymal stem cells. In another embodiment, at least one of the agents that promotes bone growth is a concentrated growth factor.

The intervertebral fusion devices preferably are fabricated by passing a solution comprising one or more agents that promote bone growth through the porous matrix of the fusion cage, thereby attaching the agent to the inner surface.

The intervertebral fusion device can be used to promote fusion of two adjacent vertebrae in a mammal, preferably a human patient. In this method, a solution that has one or more bone growth promoting agents is passed through the intervertebral fusion cage, thereby attaching the agent to the inner surface, then the fusion cage is inserted into the intervertebral space between the two vertebrae of the mammal. In a preferred embodiment, the patient's own bone marrow aspirate or platelet rich plasma is passed through the porous matrix during the surgical procedure to insert the fusion cage. The fusion cage may be inserted into the intervertebral space of a mammal from either the anterior or posterior side of the mammal using surgical techniques known to those skilled in the art.

One advantage of the intervertebral fusion device of the invention is that the device supplies a concentrated amount of mesenchymal stem cells (MSC's) from the bone marrow aspirate, and so does not require autologous bone taken from the iliac crest (which requires a second operation) or allograft bone material (which may be in limited supply). In addition, there is no risk disease transmission, as when allograft bone material is used.

### BRIEF DESCRIPTION OF THE DRAWINGS

The FIGURE is a schematic representation of a preferred embodiment of an intervertebral fusion device of the invention.

### DETAILED DESCRIPTION OF THE INVENTION

The present invention relates to a new and improved intervertebral fusion device that has a load bearing wall that can support intervertebral forces and a porous matrix having agents that promote bone growth attached the inner surface of the pores. Typically, the load bearing wall is made of a material that is denser than the porous matrix and can support a load in the range of at least about 8.2 kN (kilonewtons). This typically requires that the load bearing wall to be made of a material having a compression strength of between about 1000 MPa and 1500 MPa. In a preferred embodiment, the porous matrix is integrally bound to the load bearing wall. The fusion cage may have more than one load bearing wall. In a preferred embodiment, the fusion cage has at least two load bearing walls.

A preferred embodiment of an intervertebral fusion device (1) of the invention is seen in the FIGURE. The device has two load bearing walls (3) which are adjacent to a porous matrix (5) and an upper surface (7) and a lower surface (9) that will contact the vertebrae to be fused when the device is inserted into the intervertebral space. The device is configured so that the upper and lower surfaces are sufficiently loaded to produce bone growth.

The fusion cage can be made of any biocompatible material and has a suitable size and shape for seated implantation into the intervertebral space. In some embodiments, the load bearing wall of the fusion cage is approximately parallel to the spinal column when the device is inserted into the intervertebral space. In others, the load bearing wall is angled to produce lordosis. In one embodiment, the fusion cage has a surface at each end of the load bearing wall which will contact the two vertebrae to be fused when the cage is inserted and are approximately perpendicular to the load bearing wall. In another embodiment, the two surfaces that contact the vertebrae to be fused are not perpendicular to the load bearing wall, but instead are tapered in the anterior to posterior direction to achieve lordosis or in the posterior to anterior direction to achieve kyphosis. In a preferred embodiment, the surfaces of the fusion cage that contact the vertebrae to be fused have ridges or teeth to prevent increase the stability of the fusion device once it is inserted into the patient. Optionally, the fusion cage can be a series of cages stacked on top of each other, such as described in U.S. Pat. Nos. 6,195,211 and 5,192,327, the entire teachings of which are incorporated herein by reference.

Mechanical attachment of the porous matrix to the load bearing wall may be achieved by, for example, press fitting a porous matrix cylinder into a hollow sleeve, or by sintering to achieve an integral attachment. Preferably, the load bearing wall and the porous matrix of the fusion cage are made of the same material. However, the load bearing wall generally will have a density that is greater than the porous matrix. Typically, the load bearing wall will have a porosity in the range of between 0 and about 5 vol %, whereas the porous matrix will typically have a porosity in the range of between about 40 vol % and about 80 vol %. In a preferred embodiment, the pores have an average diameter in the range of between about 25 μm and about 1000 μm. More preferably, the average diameter of the pores is between about 100 μm and about 500 μm.

In one embodiment, the fusion cage of the invention may be made of a symthetic materical, preferably one that is stronger than bone, preferably, it is a sintered ceramic, such as an oxide of alumina, ziriconia, or a combination thereof. In ceramic materials, the pore size can be controlled by conventional techniques, such as controlling the temperature during the sintering process. Ceramic sintering techniques are known to those skilled in the art. In a preferred embodiment, the ceramic includes an osteoconductive material, such as hydroxyapatite or tricalcium phosphate, to promote bone growth into the fusion device. In one embodiment, the osteoconductive material may be added as a coating on the inner surface of the porous matrix.

Other suitable materials for the fusion cage include biopolymers such as, for example, polylactic acid, polyglycolic acid, a copolymer of polylactic acid and polyglycolic acid, a polyarylethyl ketone, polygalactic acid, polycaprolactone, polyethylene oxide, polypropylene oxide, polysulfone, polyethylene, polypropylene, a polyaryletherketone, and combinations thereof. In one embodiment, the fusion cage comprises a polyaryletherketone. In a preferred embodiment, the percentage of the fusion cage that is a polyaryletherketone is in the range of between about 40 vol % and about 90 vol %. In another embodiment, the polyaryletherketone is mixed with carbon fibers which are typically chopped. In a preferred embodiment, the percentage of the fusion cage that is carbon fiber is in the range of between about 1 vol % and about 60 vol %. Examples of polyaryletherketones include polyetheretherketone, poly(arylether ketone ketone), and polyetherketone.

An agent that promotes bone growth is attached to the inner surface of the porous matrix. Agents that promote bone growth include connective tissue progenitor cells (referred to as “progenitor cells” herein) and growth factors. The agent that promotes bone growth is attached to the inner surface, and thereby concentrated, by passing a solution containing the agent through the porous matrix one or more times. Typically, the concentration of the agent that promotes bone growth is increased in the range of between about 2 fold and about 30 fold by passing the solution through the porous matrix of the fusion cage. More preferably, the agent is increased in the range of between about 5 fold to about 20 fold, and more preferably between about 5 fold and 10 fold.

The term “progenitor cells,” as used herein, are cells that are capable of differentiating into cartilage or bone. Examples of progenitor cells include mesenchymal stem cells, hematopoietc cells, and embryonic stem cells. In one embodiment, progenitor cells are attached to the inner surface of the porous matrix by passing bone marrow aspirate suspension through the fusion cage.

As used herein, the term “growth factors” encompasses any cellular product that modulates the growth or differentiation of other cells, particularly connective tissue progenitor cells. Growth factors include, but are not limited to, isoforms of platelet derived growth factors (PDGF), fibroblast growth factors, epithelial growth factors, isoforms of transforming growth factor Beta, insulin-like growth factors, bone morphogenic proteins and precursors thereof. In a preferred embodiment, the growth factor is a bone morphogenic protein or a precursor thereof. In one embodiment, growth factors are attached to the inner surface of the porous matrix by passing platelet rich plasma or bone marrow aspirate suspension through the fusion cage. In another embodiment, growth factors are attached to the inner surface of the porous matrix by passing a solution of recombinant growth factors through the fusion cage.

Bone marrow aspirate contains plasma, nucleated connective tissue progenitor cells, nucleated hematopoietic cells, endothelial cells, and cells derived from contaminating peripheral blood, including red cells and platelets. Since bone marrow aspirate also contains peripheral blood, it is preferred that the bone marrow be collected in a syringe containing an anti-coagulant. Suitable anti-coagulants include, for example, heparin, sodium citrate, and EDTA. Preferably, the bone marrow aspirate is mixed with a sterile isotonic solution to provide a concentration in the range of from about 10 million to about 300 million nucleated cells/ml, preferably from about 20 million to about 250 million nucleated cells/ml, more preferably from about 50 million to about 200 million nucleated cells/ml. Suitable isotonic solutions include, for example, isotonic buffered salt solutions, such as Hank's Balanced Salt Solution and phosphate buffered saline, and tissue culture medium such as minimal essential medium. As used herein, the term “bone marrow aspirate suspension” refers to a bone marrow aspirate that has not been mixed with an isotonic solution and to a bone marrow aspirate that has been mixed with an isotonic solution.

Platelet rich plasma typically is produced from centrifuging blood and isolating the buffy coat produced therefrom, and may be produced from the blood of the patient receiving the intervertebral fusion device, by methods known to those skilled in the art. Platelet rich plasma contains densely concentrated platelets (which, when activated by thrombin, release growth factors).

In some embodiments, a concentrated fraction of either the BMA or PRP is passed through the porous matrix. Preferably, this concentrated fraction is the buffy coat.

To allow for implantation of the graft into a mammal, it is preferred that the fusion cage be sterile. Preferably, the bone marrow aspirate suspension is permitted to flow through the sterile fusion cage under hydrostatic pressure which may be generated by external forces or by the force of gravity. Preferably, the linear elution rate of the suspension through the fusion cage is between 2 and 500 mm/minute, more preferably between 5 and 200 mm/minute, most preferably between 10 and 100 mm/minute.

Optionally, the effluent is collected sterilely in an effluent collector and recycled through the fusion cage one or more times to increase the number of connective tissue progenitor cells attached to the inner surface of the porous matrix of the fusion cage.

In some embodiments, the device further comprises cell adhesion molecules attached to the inner surface of the porous matrix. These molecules help the device retain the agents passing therethrough.

Optionally, a wash solution is passed through the fusion cage after the original bone marrow aspirate suspension and any effluents have been passed through the fusion cage. Preferably, the wash solution comprises a sterile, isotonic, buffered solution having a pH range of 7.3 to 7.5. Suitable wash solutions include, for example, phosphate-buffered saline, Hank's balanced salt solution, and minimal essential medium.

Optionally, growth factors or additional cells which secrete or present (i.e., express on their surface) growth factors are attached to the inner surface of the porous matrix prior to use, i.e, before, during or after the time the bone marrow aspirate suspension is passed through the fusion cage. Growth factors which may be added include for example, isoforms of platelet derived growth factors, fibroblast growth factors, epithelial growth factors, transforming growth factor Beta, insulin-like growth factor(s), parathyroid hormone (PTH) or PTH related peptide, and bone morphogenic proteins and precursors thereof. Preferably, growth factors are added by passing a solution containing the growth factors through fusion cage after all previous suspensions and solutions have been passed through the substrate. Alternatively, grow factors are added by incorporation into the wash solution. Platelets, which are known to secrete growth factors and to adhere to negatively charged surfaces, are added to the graft by passing a suspension of platelets, such as blood or platelet concentrate which contains an anti-coagulant, through the fusion cage.

In devices designed for posterior lumbar interbody fusion (PLIF), the present invention is advantageous over conventional autograft-containing cages in that there is less of a chance that the graft will fall out of the cage during the high-impact insertion of the cage.

### Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. An intervertebral fusion device, comprising:
a) an intervertebral fusion cage, comprising:
i) a load bearing wall; and
ii) a porous matrix mechanically attached to the load bearing wall, wherein the open pores of the porous matrix define an inner surface; and
b) one or more agents that promote bone growth attached to the inner surface, wherein the agent is about 5 times to about 30 times more concentrated than the concentration found in bone marrow aspirate or platelet rich plasma.

2. The device of claim 1, wherein one or more of the agents that promote bone growth are a progenitor cells.

3. The device of claim 2, wherein the progenitor cells are concentrated by passing a solution of platelet rich plasma or bone marrow aspirate suspension through the porous matrix one or more times.

4. The device of claim 2, wherein the progenitor cells are selected from the group consisting of mesenchymal stem cells, hematopoietic cells, embryonic stem cells and combinations thereof.

5. The device of claim 1, further comprising a growth factor.

6. The device of claim 5, wherein the growth factor is concentrated by passing a solution comprising a recombinant growth factor through the porous matrix one or more times.

7. The device of claim 5, wherein the growth factor is concentrated by passing a solution of platelet rich plasma or bone marrow aspirate suspension through the porous matrix one or more times.

8. The device of claim 5, wherein the growth factor is selected from the group consisting of bone morphogenic protein or a precursor thereof, isoforms of platelet derived growth factors, fibroblast growth factors, epithelial growth factors, isoforms of transforming growth factor Beta, insulin-like growth factors, and combinations thereof.

9. The device of claim 8, wherein the growth factor is bone morphogenic protein or a precursor thereof.

10. The device of claim 1, wherein the fusion cage comprises a ceramic.

11. The device of claim 10, wherein the ceramic is an oxide of alumina, zirconia or a combination thereof.

12. The device of claim 11, wherein the ceramic comprises hydroxyapatite, tricalcium phosphate, or a combination thereof.

13. The device of claim 1, wherein the fusion cage comprises a biopolymer.

14. The device of claim 13, wherein the biopolymer is selected from the group consisting of polylactic acid, polyglycolic acid, a copolymer of polylactic acid and polyglycolic acid, a polyarylethyl ketone, polygalactic acid, polycaprolactone, polyethylene oxide, polypropylene oxide, polysulfone, polyethylene, polypropylene, a polyaryletherketone, and combinations thereof.

15. The device of claim 14, wherein the fusion cage comprises a polyaryletherketone selected from the group consisting of polyetheretherketone, poly(arylether ketone ketone), polyetherketone, and combinations thereof.

16. The device of claim 15, wherein the fusion cage further comprises carbon fibers.

17. The device of claim 16, wherein the composition of the fusion cage is between about 40% and about 60% polyarylether ketone.

18. The device of claim 16, wherein the composition of the fusion cage is between about 1% and about 60% carbon fiber.

19. The device of claim 1, wherein the porous matrix is integrally bound to the load bearing wall.

20. The device of claim 1, wherein the porous matrix has a porous volume of between about 40% and about 80%.

21. The device of claim 1, wherein the porous matrix has an average pore diameter of between about 25 μm and about 1000 μm.

22. The device of claim 1, wherein the load bearing wall has an upper and lower bearing surfaces that have teeth.

23. The device of claim 1, wherein the fusion cage is tapered in the anterior to posterior direction to achieve lordosis.

24. The device of claim 1, wherein the fusion cage is tapered in the posterior to anterior direction to achieve kyphosis.

25. The device of claim 1, comprising more than one fusion cage stacked on top of each other.

26. An intervertebral fusion device, comprising:
a) an intervertebral fusion cage, comprising:
i) a load bearing wall; and
ii) a porous matrix mechanically attached to the load bearing wall, wherein the open pores of the porous matrix define an inner surface; and
b) one or more agents that promote bone growth attached to the inner surface, wherein at least one agent is a concentrated growth factor.

27. A method of fabricating an intervertebral fusion device, comprising the steps of:
a) providing an intervertebral fusion cage, comprising:
i) a load bearing wall; and
ii) a porous matrix mechanically attached to the load bearing wall, wherein the open pores of the porous matrix define an inner surface; and
b) passing a solution comprising one or more agents that promote bone growth through the porous matrix, thereby attaching the agent to the inner surface.

28. The method of claim 27, wherein the solution comprises progenitor cells.

29. The method of claim 28, wherein the progenitor cells are selected from the group consisting of mesenchymal stem cells, hematopoietc cells, embryonic stem cells, and combinations thereof.

30. The method of claim 28, wherein the solution is platelet rich plasma or bone marrow aspirate suspension.

31. The method of claim 27, wherein the solution comprises a growth factor.

32. The method of claim 31, wherein the growth factor is selected from the group consisting of bone morphogenic protein or a precursor thereof, isoforms of platelet derived growth factors, fibroblast growth factors, epithelial growth factors, isoforms of transforming growth factor Beta, insulin-like growth factors, and combinations thereof.

33. The method of claim 32, wherein the growth factor is bone morphogenic protein or a precursor thereof.

34. The method of claim 31, wherein the solution comprises a recombinant growth factor.

35. The method of claim 31, wherein the solution is platelet rich plasma or bone marrow aspirate suspension.

36. A intervertebral fusion device fabricated using the method of claim 27.

37. A method of promoting fusion of two consecutive vertebrae in a mammal, comprising the steps of:
a) providing an intervertebral fusion cage, comprising:
i) a load bearing wall; and
ii) a porous matrix mechanically attached to the load bearing wall, wherein the open pores of the porous matrix define an inner surface; and
b) passing a solution comprising one or more agents that promote bone growth through the porous matrix, thereby attaching the agent to the inner surface; and
c) inserting the fusion cage containing the agent into the intervertebral space between the two vertebrae.

38. The method of claim 37, wherein the solution comprises progenitor cells.

39. The method of claim 38, wherein the progenitor cells are selected from the group consisting of mesenchymal stem cells, hematopoietc cells, embryonic stem cells, and combinations thereof.

40. The method of claim 38, wherein the solution is platelet rich plasma or bone marrow aspirate suspension.

41. The method of claim 40, wherein the solution is an autologous solution.

42. The method of claim 41, wherein the solution is passed through the porous matrix during a surgical procedure to insert the fusion cage.

43. The method of claim 37, wherein the solution comprises a growth factor.

44. The method of claim 43, wherein the growth factor is selected from the group consisting of bone morphogenic protein or a precursor thereof, isoforms of platelet derived growth factors, fibroblast growth factors, epithelial growth factors, isoforms of transforming growth factor Beta, insulin-like growth factors, and combinations thereof.

45. The method of claim 44, wherein the growth factor is bone morphogenic protein or a precursor thereof.

46. The method of claim 43, wherein the solution comprises a recombinant growth factor.

47. The method of claim 44, wherein the solution is platelet rich plasma or bone marrow aspirate suspension.

48. The method of claim 47, wherein the solution is an autologous solution.

49. The method of claim 48, wherein the solution is passed through the porous matrix during a surgical procedure to insert the fusion cage.

50. The method of claim 37, wherein the fusion cage is inserted from the anterior side of the mammal.

51. The method of claim 37, wherein the fusion cage is inserted from the posterior side of the mammal.
